(19) United States
(12) Patent Application Publication
(10) Pub. No.: US 2007/0270694 A1
(43) Pub. Date: Nov. 22, 2007

(54) MODULAR DIGITAL ULTRASOUND IMAGING SYSTEM

(76) Inventors: Laurent PELISSIER, Vancouver (CA); Sergio BERNARDO, Vancouver (CA); Paul SIDLICK, Port Moody (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 11/670,940

(22) Filed: Feb. 2, 2007

Related U.S. Application Data
(60) Provisional application No. 60/744,194, filed on Apr. 3, 2006.
(60) Provisional application No. 60/744,192, filed on Apr. 3, 2006.
(60) Provisional application No. 60/744,193, filed on Apr. 3, 2006.

(51) Int. Cl. A61B 8/00

(52) U.S. Cl. 600/443

(57) ABSTRACT

A housing unit for an ultrasound system which comprises an ultrasound transducer, a video display, an input device and a removable PC computer. The removable PC may be housed within an enclosure defined by the housing unit. The removable PC may house all of the processor(s) required for control and operation of the ultrasound system. The removable PC may be coupled to and uncoupled from the ultrasound transducer, the video display and the input device using conventional connectors. Such connectors permit users to interchange the removable PC with an updated PC without having to send the ultrasound system to the manufacturer and without requiring the services of a technician.

### CROSS-REFERENCE TO RELATED APPLICATIONS

This application claims priority from U.S. provisional patent application No. 60/744,194 entitled MODULAR DIGITAL ULTRASOUND IMAGING SYSTEM filed 3 Apr. 2006. This application is related to the following co-owned U.S. patent applications:
(i) application No. 60/744,192 entitled METHODS AND SYSTEMS FOR TRANSFIGURING ULTRASOUND SYSTEMS FOR ULTRASOUND EXAMINATION filed 3 Apr. 2006; and
(ii) application No. 60/744,193 entitled ULTRASONIC IMAGING SYSTEM HAVING COMPUTER COUPLED TO RECEIVE AND PROCESS DATA filed 3 Apr. 2006
   All three of these related applications are hereby incorporated by reference herein.

### TECHNICAL FIELD

The invention applies to digital ultrasound imaging systems.

Particular embodiments of the invention provide modular housing units for digital ultrasound imaging systems.

### BACKGROUND

Ultrasound technology has a wide variety of medical applications related to exploration and imaging of internal regions of a patient's body. Ultrasound imaging is generally non-destructive and versatile and can provide high quality images useful for medical examination, diagnosis of medical conditions and the like.

Ultrasound imaging systems typically comprise: an ultrasound transducer which incorporates an array of transducer elements for sensing information (in the form of acoustic signals) from the body of a patient; and a display unit for displaying information representative of the signals received by the transducer. Recent developments have led to the introduction of digital ultrasound imaging systems. Digital ultrasound systems sample and digitize ultrasound data that is captured by the ultrasound transducer. Digital ultrasound systems are typically controlled and/or controllable (at least in part) by software that runs on one or more processor(s), such as may be provided by custom embedded hardware systems and/or embedded hardware systems in combination with a personal computer (PC), for example. Examples of digital ultrasound imaging systems include those disclosed in U.S. Pat. No. 6,325,759 (Pelissier), U.S. Pat. No. 5,839,442 (Chiang et al.), U.S. Pat. No. 5,795,297 (Daigle) and U.S. Pat. No. 5,758,649 (Iwashita et al.), all of which are hereby incorporated herein by reference.

In addition to the ultrasound transducer and the video display, a digital ultrasound system includes one or more suitably programmed processor(s) which execute software instructions to control operation of the ultrasound system. Some digital ultrasound systems incorporate custom hardware which includes such suitably programmed processor(s). These systems suffer from the drawback that they are not easily updated or repaired by users (i.e. users, such as doctors, nurses or other medical professionals, are unlikely to be able to perform hardware or software upgrades or repairs). This disadvantage is particularly problematic because of the relatively rapid rate of increase in hardware processing power and corresponding software processing techniques. Increases in hardware processing power and corresponding software processing techniques can lead to new and useful ultrasound imaging modalities. Such new ultrasound imaging modalities may not be supported by the original custom hardware and software (i.e. upgrades may be required to take advantage of such new ultrasound imaging modalities). Consequently, ultrasound imaging systems which incorporate custom hardware may not be able to take advantage of the latest ultrasound techniques. In some circumstances, ultrasound imaging systems may have to be sent out from a medical facility or otherwise rendered unavailable for use, so that they can be serviced and/or updated by their manufacturer.

Another disadvantage of ultrasound systems incorporating custom hardware relates to the use of such systems in medical environments. Medical environments, such as hospitals, are typically subject to strict regulations regarding the equipment used therein. Such equipment must often meet safety standards set by policies of the hospital itself and/or by various regulatory authorities. An example requirement for hospital equipment is to maintain electrical and magnetic field (EMF) emissions below an acceptable threshold. System updates which involve changes to the custom hardware of an ultrasound system (typically requiring service by the manufacturer of the ultrasound system) may require that the entire ultrasound system be re-certified to ensure that it meets with regulatory approval.

Some digital ultrasound systems incorporate a combination of custom hardware (typically used for high speed processing or preprocessing of ultrasound signals received from the transducer) and conventional PC hardware (typically used for providing a user interface and for controlling the custom hardware). Systems which incorporate custom processing or preprocessing hardware may suffer from the same drawbacks as those systems having completely custom hardware. More specifically, systems which incorporate custom processing or preprocessing hardware are not easily user-updatable or user-repairable and such systems may not be able to take advantage of the latest ultrasound techniques.

Ultrasound systems incorporating a desktop PC may not be easily mobile. Mobility is advantageous in a hospital environment, where patients may not be mobile and where equipment and space are scarce. Where ultrasound systems incorporate a laptop PC, the PC may be susceptible to theft and/or damage. Also, laptop PCs are often insufficiently robust for use in a hospital environment where they may be handled by a large number of users. Laptop PCs may also emit unacceptable levels of EMF.

There is a general desire to provide digital ultrasound systems that overcome or at least ameliorate one or more of the aforementioned drawbacks with prior art systems.

### SUMMARY OF THE INVENTION

One aspect of the invention provides a housing unit for an ultrasound system which comprises an ultrasound transducer, a video display, an input device and a removable PC computer. The removable PC may be housed within an enclosure defined by the housing unit. The removable PC may house all of the processor(s) required for control and operation of the ultrasound system. The removable PC is preferably capable of being coupled to and uncoupled from the ultrasound transducer, the video display and the input device using conventional connector interfaces. Such connectors permit users to interchange the removable PC with an updated PC (i.e. a PC having updated software and/or hardware) without having to send the ultrasound system to the manufacturer and without requiring the services of a technician. Such connectors permit users to replace damaged or faulty PCs without having to send the ultrasound system to the manufacturer and without requiring the services of a technician. In some embodiments, the housing unit can obtain regulatory certification separately from the PC, such that updates to the PC and/or replacement of the PC does not require separate regulatory certification of the system. Such certification may involve the amount of EMF generated by the system, for example.

Further features and applications of the invention are described below.

### BRIEF DESCRIPTION OF THE DRAWINGS

In drawings which depict non-limiting embodiments of the invention:

FIG. 1 is a front isometric view of a housing unit for a digital ultrasound system according to a particular embodiment of the invention;

FIG. 2A is a rear isometric view of the FIG. 1 housing unit with its PC-enclosure in a closed configuration;

FIG. 2B is a rear isometric view of the FIG. 1 housing unit with its PC-enclosure in an open configuration;

FIG. 3A is a schematic block diagram showing the components of a digital ultrasound system according to a particular embodiment of the invention;

FIG. 3B is a schematic block diagram showing the components of a digital ultrasound system according to another embodiment of the invention;

FIG. 4A is a perspective view of a PC-enclosure of the FIG. 1 housing unit with the PC removed;

FIG. 4B is an isometric view of a PC suitable for use with the housing unit and ultrasound system of FIG. 1;

FIGS. 5A, 5B and 5C depict isometric views of a housing unit for a digital ultrasound system according to another embodiment of the invention.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

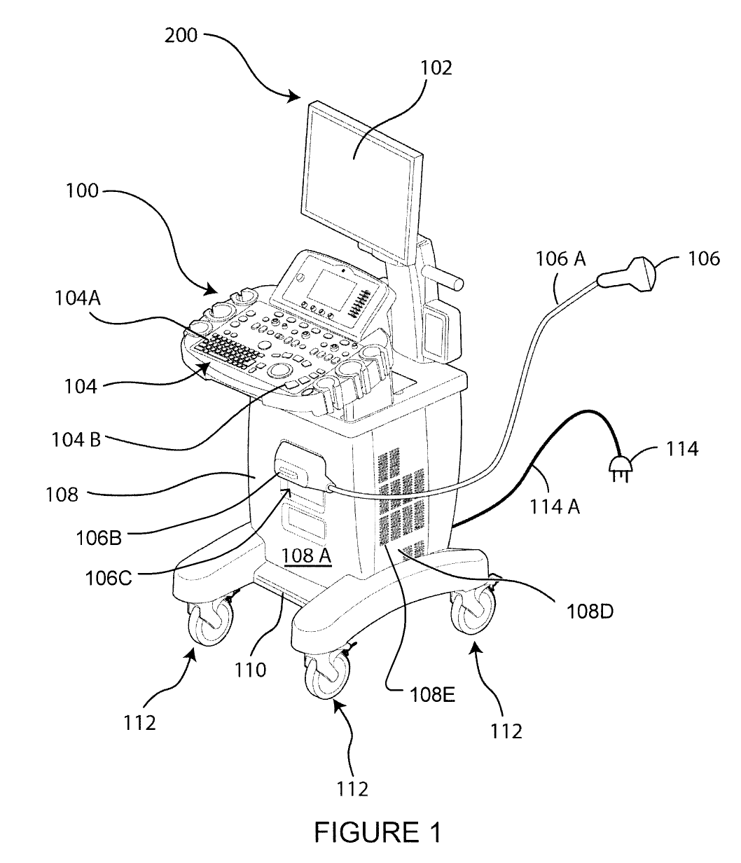

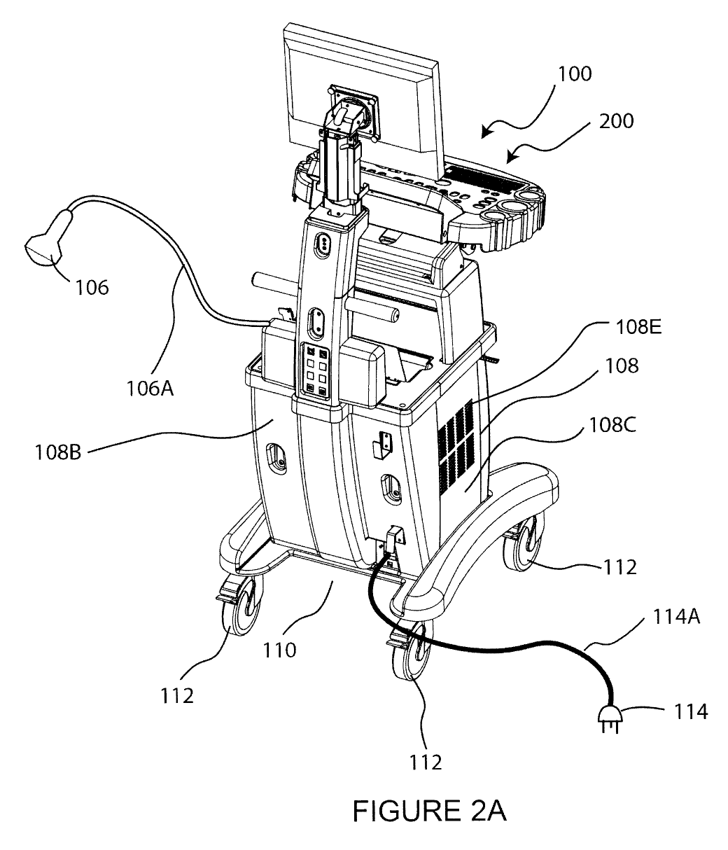

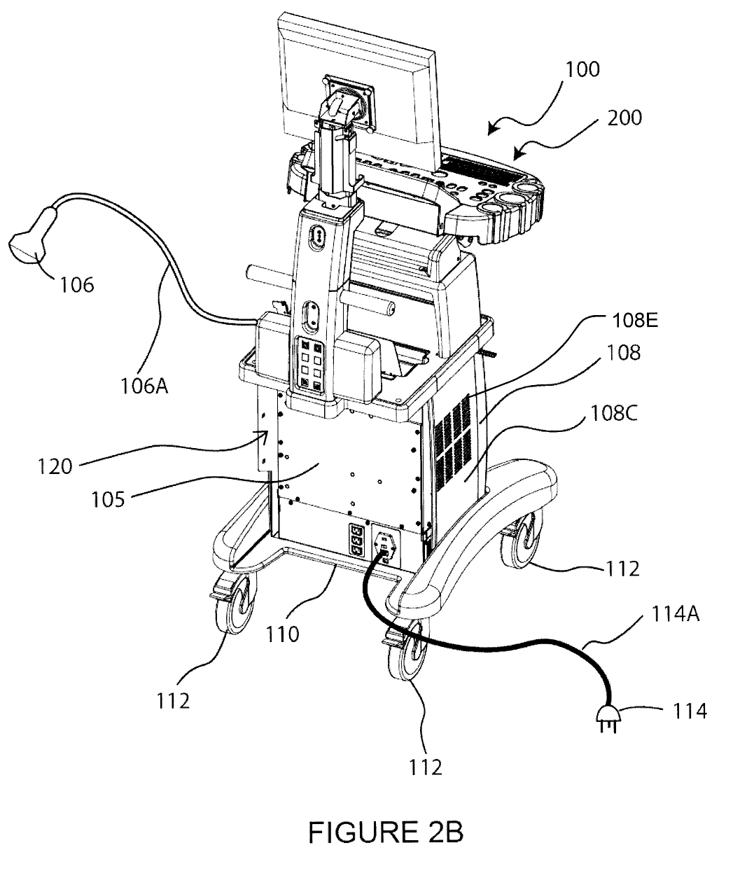

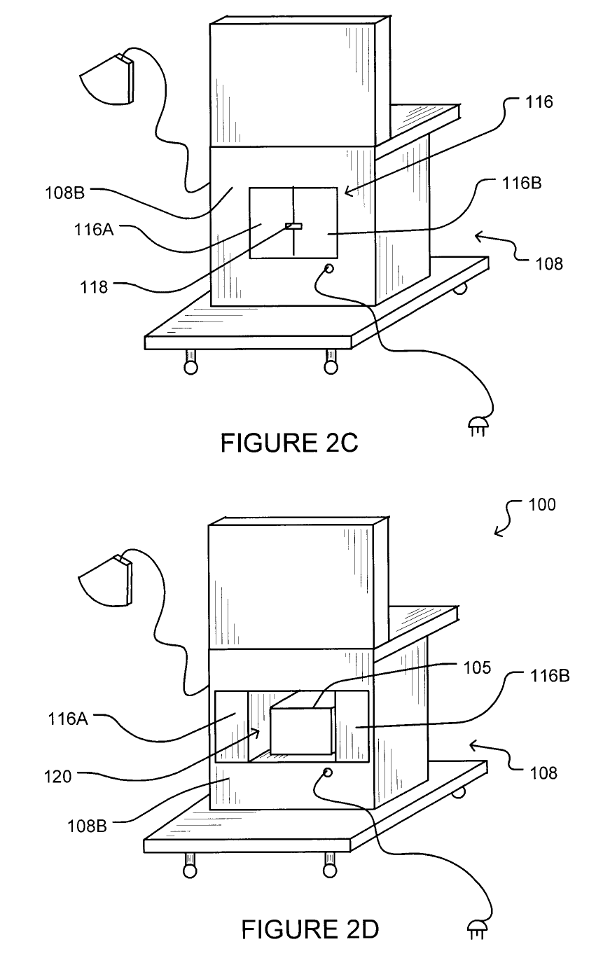

FIGS. 1, 2A and 2B show different views of a housing unit 100 for a modular digital ultrasound system 200 according to a particular embodiment of the invention. System 200 comprises a video display 102, an input device 104, a transducer 106 and a PC 105. Preferably, video display 102 is a high resolution, color, flat-screen monitor. In general, however, video display 102 may comprise any other type(s) of video display(s) known in the art. In the illustrated embodiment, input device 104 comprises a keyboard 104A with customized function keys 104B. PC 105 may comprise a self-contained personal computer having its own case, power supply, processor, memory and ports. In some embodiments, system 200 may comprise one or more additional input devices (not shown in FIG. 1), such as a pointing device (e.g. a mouse) or the like.

Transducer 106 typically comprises an array of piezoelectric transducers (not shown). Transducer 106 generates acoustic signals which are directed into an object to be imaged, such as the body of a patient. These acoustic signals are reflected from the patient's body as acoustic echo signals. The acoustic echo signals are received by transducer 106 and, in response to the acoustic echo signals, transducer 106 generates electrical echo signals. Ultrasound transducers (e.g. transducer 106) are known in the art. U.S. Pat. No. 5,839,442 (Chiang et al.) provides a description of the components and operation of one example ultrasound transducer.

In the illustrated embodiment, all of the components of ultrasound imaging system 200 are contained in housing unit 100 except for transducer 106, which is tethered to housing unit 100 by transducer cord 106A and transducer interface 106B, and plug 114, which provides power to housing unit 100 via power cord 114A. Housing unit 100 comprises a stand portion 108. Stand portion 108 may comprise a transducer port 106C for receiving transducer interface 106B. In the illustrated embodiment, stand portion 108 supports video display 102 and input device 104, although this is not necessary. Stand portion 108 may itself be supported by (and releasably or fixedly coupled to) a mobile cart 110. Mobile cart 110 may comprise a plurality of castors 112, wheels or the like which allow cart 110 to move about on a floor. In some embodiments, castors 112 may be mounted to the bottom surface of stand portion 108, in which case cart 110 may not be required.

As shown in FIGS. 2A and 2B, stand portion 108 incorporates a PC-enclosure 120 which accommodates PC 105. In the illustrated embodiment PC-enclosure 120 is accessed via a removable rear wall 108B of stand 108. Rear wall 108B may be removably secured to stand portion by a suitable latch mechanism, which may be a locking latch mechanism. In other embodiments, the access to PC-enclosure 120 may be provided by another one or more of the walls 108A, 108B, 108C, 108D of stand portion 108 or by an openable and/or removable cover portion of one or more of the walls 108A, 108B, 108C, 108D of stand portion 108. In the illustrated embodiment, side walls 108C and 108D are perforated by one or more apertures 108E which allow for a flow of air therethrough. Such air may be used to cool PC-enclosure 120 and PC 105.

FIGS. 2C and 2D show an embodiment with a cover 116 located in rear wall 108B of stand 108. Cover 116 comprises a pair of hinged doors 116A, 116B. Doors 116A, 116B are pivotable about hinges (not shown), such that PC-enclosure 120 is configurable between a closed configuration (FIG. 2C), where PC 105 is enclosed in enclosure 120, and an open configuration (FIG. 2D), where PC 105 is accessible. In some embodiments, cover 116 may be perforated by one or more apertures (not shown) which allow for a flow of air therethrough. Such air may be used to cool PC-enclosure 120 and PC 105.

In the embodiment of FIGS. 2C and 2D, doors 116A, 116B each comprise a portion of a latch mechanism 118, which latches doors 116A, 116B in the closed configuration of FIG. 2C. Preferably, latch mechanism 118 may be locked using a conventional lock (not shown). In other embodiments, cover 116 comprises a single hinged door. In still other embodiments, cover 116 is slidable relative to stand portion 108 and/or removable from stand portion 108, such that PC-enclosure 120 is configurable between open and closed configurations. Suitable latch mechanisms (preferably lockable latch mechanisms) may be provided for such other cover embodiments.

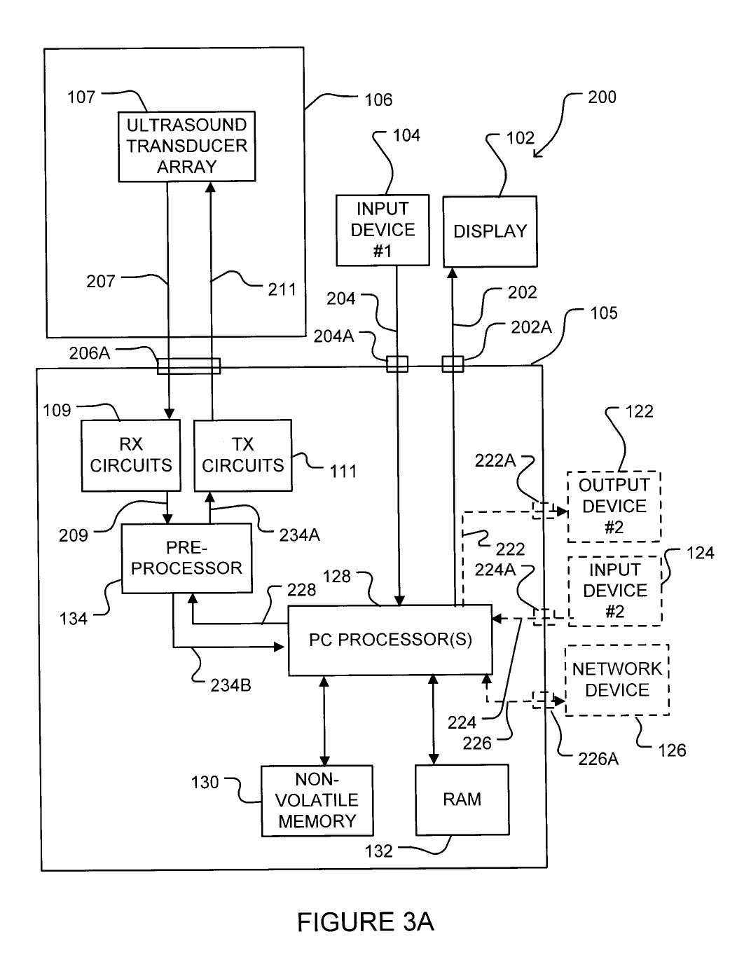

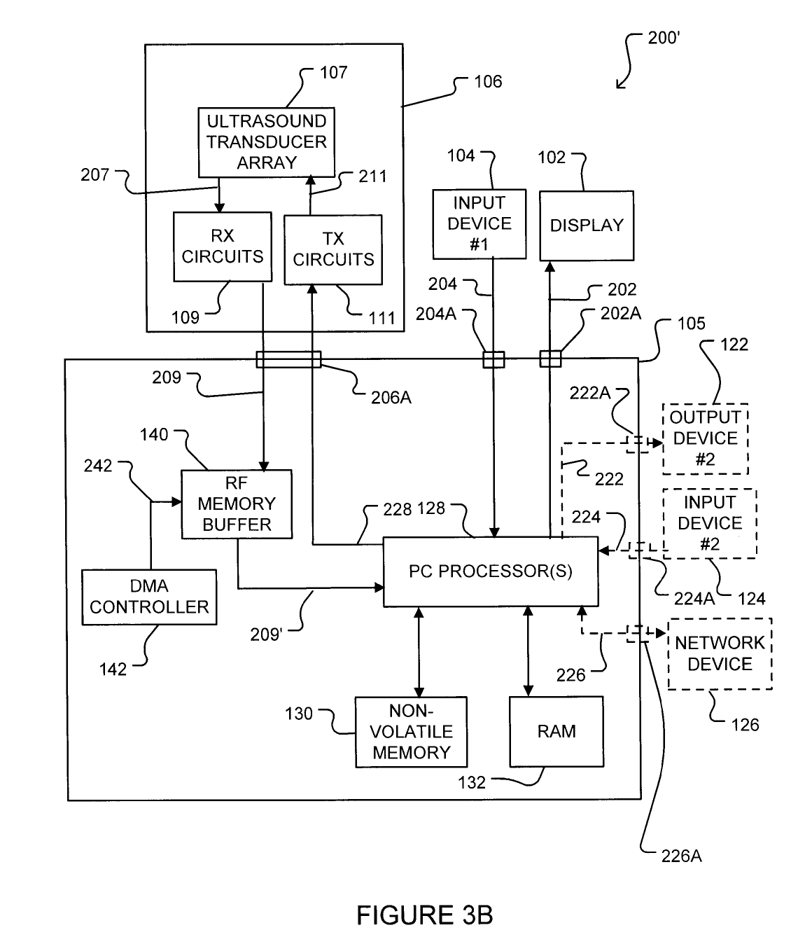

FIG. 3A schematically depicts a digital ultrasound system 200 according to a particular embodiment of the invention. Ultrasound system 200 comprises video display 102, input device 104, ultrasound transducer 106 and PC 105. Video display 102 and input device 104 are preferably used to provide a graphical user interface. In the illustrated embodiment, ultrasound transducer 106 comprises a transducer array 107, which is connected to receive circuits 109 and transmit circuits 111 located within PC 105 via an interface 206A. Interface 206A may be a custom connector or a standard digital connector. In other embodiments, receive circuits 109 and transmit circuits 111 are provided by cards, circuit boards or the like, located within transducer 106 or external to both transducer 106 and PC 105. Receive circuits 109 may comprise amplifiers, noise reduction filters and analog to digital converters (ADCs). Transmit circuits 111 may comprise signal drivers and the like.

In the illustrated embodiment, PC 105 comprises one or more main PC processors 128, which communicate with RAM 132 and with non-volatile memory 130 (e.g. a hard drive). PC 105 of system 200 also comprises ultrasound signal preprocessing hardware 134. Preprocessing hardware 134 may be implemented on one or more cards, circuit boards or the like located within PC 105. In some embodiments, preprocessing hardware 134 is located external to PC 105. In some embodiments, receive circuits 109 and transmit circuits 111 are implemented as a part of preprocessing hardware 134. An example of suitable preprocessing hardware is describe in U.S. Pat. No. 6,325,759 (Pelissier).

In operation, a user operates input device 104 to provide a number of control instructions. In response to these control instructions, input device 104 generates input signal 204, which is received at PC 105 and ultimately by PC processor(s) 128 via interface 204A. In the case where input device 104 is a keyboard, interface 204A may be a 5-pin Deutsche Industrie Norm (DIN) connector, a 6-pin mini-DIN (PS2) connector or a Universal Serial Bus (USB) connector, for example. System 200 may incorporate an optional additional input device 124 which may provide an additional input signal 224 to PC processor(s) 128 via interface 224A. Optional additional input device 124 may comprise a mouse, a CD drive, a floppy disk drive, a pointing device, or the like. Where optional additional input device 124 is a mouse, interface 224A may be DB9 serial port connector, a 6-pin mini-DIN (PS2) connector or a USB connector.

In response to receiving input signal 204, PC processor(s) 128 may commence an ultrasound imaging operation by sending configuration data signal 228 to pre-processing hardware 134. Pre-processing hardware 134 interprets configuration data signal and outputs appropriate transducer drive signal 234A to transmit circuits 111. Transmit circuits 111 amplify transducer drive signal 234A and provide the resultant amplified transducer drive signal 211 from PC 105 to transducer array 107 via interface 206A. One or more of the individual transducers of array 107 then output acoustic signals as directed by amplified transducer drive signal 211.

The acoustic signal(s) emitted from transducer array 107 reflect from the patient's body to result in acoustic echo signals. One or more of the individual transducers in array 107 detect these echo signals and convert them to electrical echo signals 207. Electrical echo signals 207 are provided from transducer array to receive circuits 109 in PC 105 via interface 206A. Receive circuits 109 convert electrical echo signals 207 to digital echo signals 209. Digital echo signals 209 may be referred to as “RF echo data” 209 or simply “RF data” 209. RF data 209 are provided to pre-processing hardware 134. In the illustrated embodiment, transmit circuits 111 communicate amplified transducer drive signal 211 to transducer array 107 via the same interface 206A as receive circuits 109 receive electrical echo signals 207 from transducer array 107. In other embodiments, two separate interfaces may be used for these purposes.

Preprocessing hardware 134 processes digital echo signals 209 to generate preprocessed data 234B which is provided to PC processor(s) 128. PC processor(s) 128 perform additional processing on preprocessed data 234B to generate an output signal 202. Output signal 202 may be provided to display 102 via interface 202A. Interface 202A may comprise an HD15 connector, a DB9 or DB15 connector, a 13W3 connector, a four-pin mini-DIN connector, a DVI connector, one or more BNC connectors, one or more RCA connectors, or any other video connector. Display 102 outputs output signal 202 in the form of a video display that is viewable by users. PC processors 128 may also provide output signal 202 to other devices. Such other devices may include: non-volatile memory 130 (e.g. a hard drive) where output signal 202 can be recorded; and optional additional output device(s) 122 (e.g. a printer, a CD writer or the like), where the results of an ultrasound imaging operation can be outputted in a different format via signal 222 and interface 222A.

Ultrasound system 200 may optionally incorporate a network device 126 for connecting to other digital devices (e.g. a router, an ethernet adapter or wireless internet adapter or the like). Signals 226 may be input and/or output between PC 105, PC processor(s) 128 and network device 126 via interface 226A.

In some embodiments, software updates for system 200 (e.g. updates to the software operating on PC processor(s) 128 and/or preprocessing hardware 134) may be updated via signals 226 received from network device 126. For example, new software may be downloaded from a server that is connected to the same network as network device 126. In other embodiments, software updates for system 200 may be provided via optional additional input device 124. For example, updates to the software may be read from a CD.

FIG. 3B schematically depicts a digital ultrasound system 200′ according to another embodiment of the invention. In many respects, digital ultrasound system 200′ is similar to system 200 of FIG. 3A, except that system 200′ is implemented without preprocessing hardware 134, and receive and transmit circuits 109 and 111 are located in transducer 106. In system 200′, PC processor(s) 128 use the information from input signal 204 to generate a transducer drive signal 228 which is provided directly to transmit circuits 111 (via interface 206A) without preprocessing. RF data 209 from receive circuits 109 is buffered in memory buffer 140 (via interface 206A) without preprocessing. Direct Memory Access (DMA) controller 142 provides a signal 242 to buffer 140 which causes buffer 140 to release RF data 209′ to PC processor(s) 128 at a rate that PC processor(s) 128 can handle.

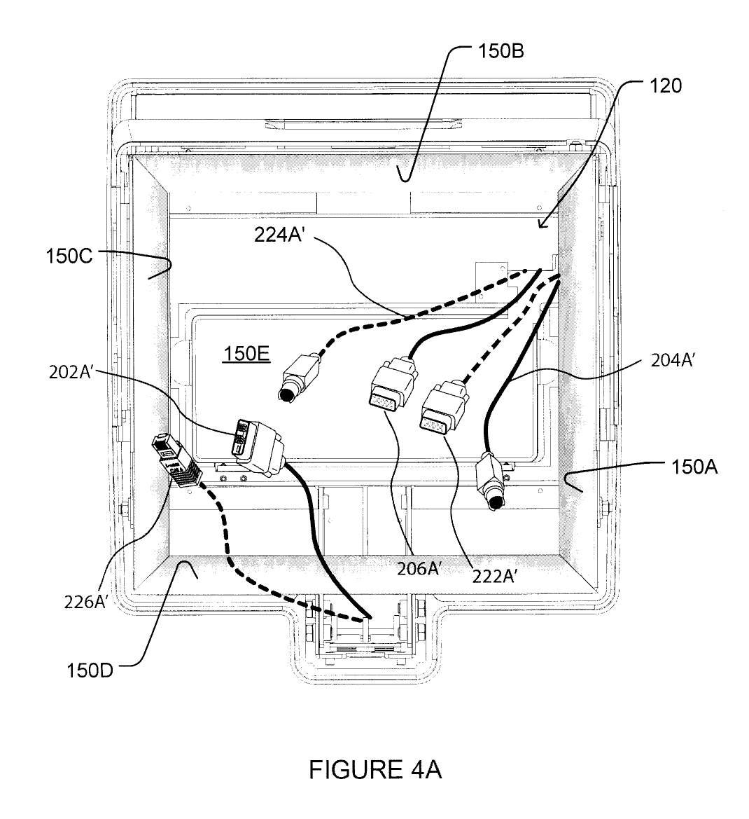

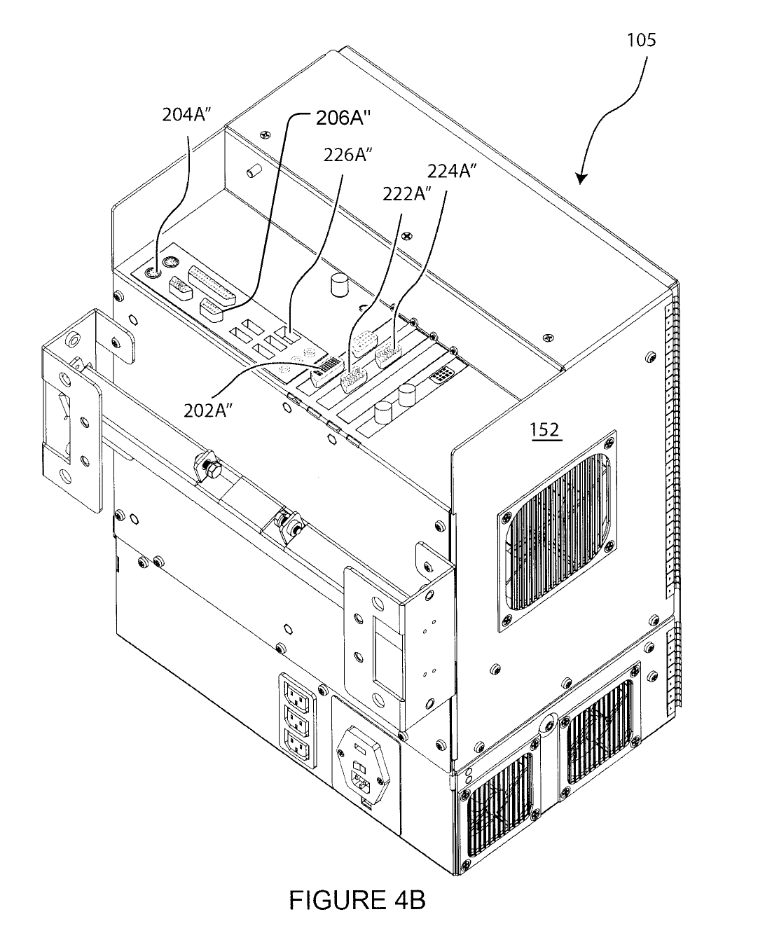

FIGS. 4A and 4B respectively depict PC-enclosure 120 (with rear wall 108B removed)of housing unit 100 and PC 105 of system 200 of the embodiment of FIGS. 1, 2A and 2B. In the illustrated embodiment, enclosure 120 is defined by rear wall 108B (not shown in FIG. 4A) and walls 150A, 150B, 150C, 150D, 150E (collectively, walls 150). Rear wall 108B and/or walls 150 may be apertured to allow for the flow of cooling air to PC 105. PC 105 may be housed in a case 152. Case 152 may also be apertured to allow for the flow of cooling air therethrough. Case 152 may be supported by legs (not shown) which provide clearance between case 152 and wall 150D of enclosure 120. In some embodiments, suitable brackets (not shown) may be used to fasten PC 105 in place within enclosure 120.

A number of connector components are contained in enclosure 120 and a number of corresponding (i.e. mating) connector components are accessible through case 152 of PC 105 (e.g. on suitable cards, such as PCI cards, for example). More specifically, in the illustrated embodiment:
display 102 is connected to PC 105 via connector 202A (FIGS. 3A, 3B) and connector 202A is implemented by connector component 2A′ (in enclosure 120) and mating connector component 202A″ (in PC 105);
input device 104 is connected to PC 105 via connector 204A (FIGS. 3A, 3B) and connector 204A is implemented by connector component 4A′ (in enclosure 120) and mating connector component 204A″ (in PC 105); and
ultrasound transducer 206 is connected to PC 105 via connector 206A (FIGS. 3A, 3B) and connector 206A is implemented by connector component 206A′ (in enclosure 120) and mating connector component 6A″ (in PC 105).

In addition, system 200 may comprise:
optional additional output device 122 which may be connected to PC 105 via connector 222A and connector 222A may be implemented by connector component 222A′ (in enclosure 120) and mating connector component 222A″ (in PC 105);
optional additional input device 124 which may be connected to PC 105 via connector 224A and connector 224A may be implemented by connector component 224A′ (in enclosure 120) and mating connector component 224A″ (in PC 105); and
optional network device 126 which may be connected to PC 105 via connector 226A and connector 226A may be implemented by connector component 226A′ (in enclosure 120) and mating connector component 226A″ (in PC 105).

In some embodiments, optional additional output device 122 and/or optional additional input device 124 may be implemented by a floppy disc drive, a CD drive or the like (not shown) in which case, connectors 222A, 222B may be located within case 152.

Advantageously, in the illustrated embodiments of ultrasound system 200 and ultrasound system 200′, PC 105 incorporates all of the processing hardware (i.e. processor(s) 128 and preprocessor 134 in system 200). As such, if system 200, 200′ requires a hardware or software upgrade, such upgrade can be provided by simply unplugging the connector components contained in enclosure 120 from the connector components in PC 105, replacing PC 105 with a newly upgraded PC and re-plugging the connector components. This type of unplugging and re-plugging of common connector components can be performed by the operators of ultrasound system 200, 200′ (i.e. without requiring special service personnel). If a software upgrade only is required, then no disconnection of PC 105 is required and the new software can be introduced via network device 126 and/or additional input device 124.

In a hospital situation, the reliability of ultrasound system 200, 200′ may be critical. For example, the availability of system 200, 200′ may be a matter of life and death. Consequently, it may be desirable for the hospital to stock a replacement PC, such that if the original PC 105 breaks down, the replacement PC may be substituted in its place. In some embodiments (e.g. where preprocessor 134 is located external to PC 105 or where DMA controller 142 and memory buffer 140 are located external to PC 105 or where such hardware is not required, any PC may be used to replace PC 105 if PC 105 breaks down. For example, a user may simply connect his or her laptop to the connector components 202A′, 204A′, 206A′ and any of the optional connector components 222A′, 224A′, 226A′ in enclosure 120, load appropriate software onto their laptop and perform ultrasound imaging operations.

In some circumstances, housing 100 can be made to pass hospital safety regulations regardless of a change in PC 105. Advantageously, this could allow PC 105 to be replaced and/or updated without having to subject the entire ultrasound system 200, 200′ to additional safety testing. For example, housing 100 may provide shielding against EMF emitted by PC 105. In some circumstances, the shielding effect of housing 100 may be certified independently of PC 105 such that PC 105 can be replaced and/or updated without having to subject the entire ultrasound system 200, 200′ to additional safety testing.

Housing 100 and ultrasound systems 200, 200′ housed therein are mobile and can be moved in circumstances where a patient is immobile.

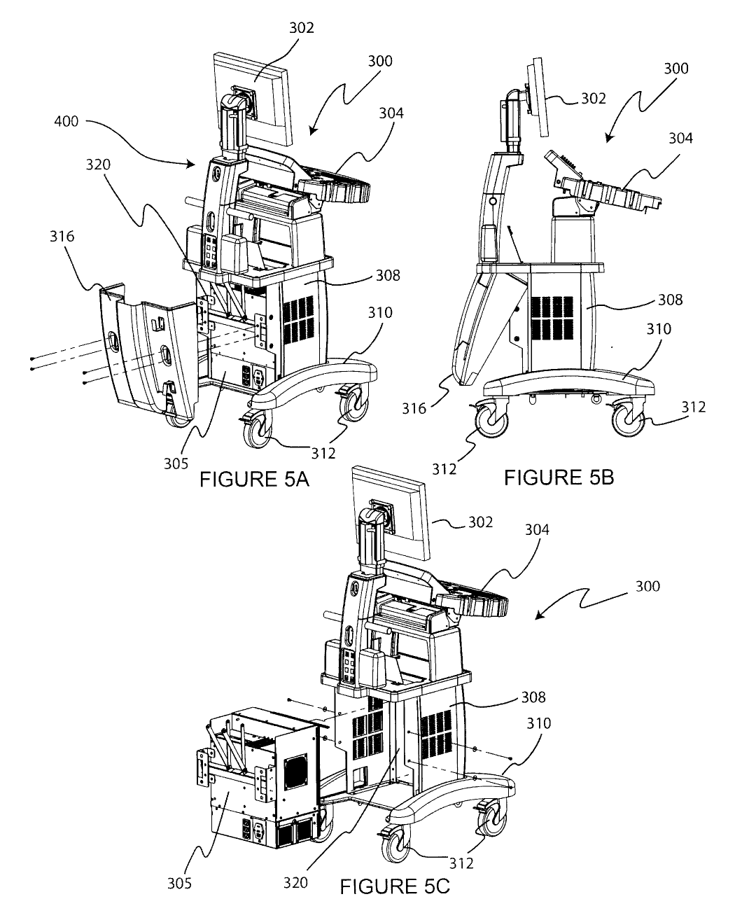

FIGS. 5A-5C show different views of a housing unit 300 for a modular digital ultrasound system 400 according to another embodiment of the invention. In many respects, housing unit 300 and system 400 are similar to housing unit 100 and system 200 described above. Reference numerals used to reflect components of housing unit 300 and ultrasound system 400 that are similar to components of housing unit 100 and ultrasound system 200 are preceded by a “3” or “4” rather than a “1” or “2” as the case may be.

System 400 comprises a video display 302, an input device 304, a transducer (not shown) and a PC 305. In the illustrated embodiment, input device 304 comprises a keyboard. As with system 200, all of the components of ultrasound imaging system 400 are contained in housing unit 300 except for the transducer, which is tethered to housing unit 300 by a transducer cord (not shown), and a plug (not shown), which provides power to housing unit 300.

Housing unit 300 comprises a stand portion 308. In the illustrated embodiment, stand portion 308 supports video display 302 and input device 304, although this is not necessary. Stand portion 308 may itself be supported by (and releasably or fixedly coupled to) a mobile cart 310. Mobile cart 310 may comprise a plurality of castors 312, wheels or the like which allow cart 310 to move about on a floor.

Stand portion 308 incorporates a PC-enclosure 320 which accommodates PC 305. PC-enclosure 320 is accessed via removable cover 316. In some embodiments, the cover 316 is perforated by one or more apertures (not shown) which allow for a flow of cooling air therethrough.

As will be apparent to those skilled in the art in the light of the foregoing disclosure, many alterations and modifications are possible in the practice of this invention without departing from the spirit or scope thereof. For example:
In some embodiments, DMA controller 142 and/or buffer 140 can be provided external to PC 105.
In some embodiments, pre-processing hardware 134 may be provided external to PC 105.
Although a tower-type PC is shown in the illustrated example embodiments, in other embodiments other types of data processing apparatus may be inserted into enclosure 120, such as slim computers, laptop computers, or any other suitable data processing apparatus.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A housing apparatus for an ultrasound system comprising a display, an input device, a transducer, and a data processing apparatus, the housing apparatus comprising:
a stand portion defining an enclosure for removably receiving the data processing apparatus;
a display connector component located in the enclosure for coupling the data processing apparatus to the display;
an input device connector component located in the enclosure for coupling the data processing apparatus to the input device; and,
a transducer connector component located in the enclosure for coupling the data processing apparatus to the transducer; whereby the ultrasound system may be upgraded or repaired by removing the data processing apparatus from the enclosure and inserting a different data processing apparatus in the enclosure.

2. A housing apparatus according to claim 1 wherein the stand portion is configured to provide shielding against electromagnetic fields emitted within the enclosure.

3. A housing apparatus according to claim 2 wherein the display and the input device of the ultrasound system are mounted on the stand portion.

4. A housing apparatus according to claim 3 comprising a cart which supports the stand portion.

5. A housing apparatus according to claim 4 wherein the cart is mounted on castors to facilitate movement of the housing apparatus.

6. A housing apparatus according to claim 2 comprising a cover for providing access to the enclosure.

7. A housing apparatus according to claim 6 wherein the cover comprises a removable wall of the stand portion.

8. A housing apparatus according to claim 6 wherein the cover comprises a hinge which allows pivotable movement of the cover between a closed position and an open position.

9. A housing apparatus according to claim 8 comprising a latch for securing the cover in the closed position.

10. A housing apparatus according to claim 1 wherein the stand portion comprises a transducer port for receiving a transducer interface coupled to the transducer, the transducer interface connected to the transducer connector component.

11. A housing apparatus according to claim 2 comprising one or more additional connector components located in the enclosure for coupling the data processing apparatus to one or more of an additional input device, an additional output device, and a network device.

12. An ultrasound apparatus comprising:
a stand portion defining an enclosure;
a data processing apparatus removably mounted in the enclosure;
a display mounted on the stand portion and coupled to the data processing apparatus by a display connector extending into the enclosure;
an input device mounted on the stand portion and coupled to the data processing apparatus by an input device connector extending into the enclosure; and,
a transducer tethered to the stand portion by a transducer cord and coupled to the data processing apparatus by a transducer connector extending into the enclosure,
whereby the ultrasound apparatus may be upgraded or repaired by uncoupling the display, input device and transducer connectors from the data processing apparatus, removing the data processing apparatus from the enclosure, inserting a different data processing apparatus in the enclosure and coupling the display, input device and transducer connectors to the different data processing apparatus.

13. An ultrasound apparatus according to claim 12 wherein the enclosure is electromagnetically shielded.

14. An ultrasound apparatus according to claim 12 wherein the data processing apparatus comprises a power supply, a memory and a processor contained in a case.

15. An ultrasound apparatus according to claim 12 wherein the data processing apparatus comprises a computer.

16. An ultrasound apparatus according to claim 15 wherein the computer comprises memory storing instructions for receiving data from the transducer and displaying an image on the display based on the received data.

17. An ultrasound apparatus according to claim 15 wherein the computer comprises a self-contained personal computer comprising a case, a power supply, a processor, memory, and a plurality of ports.

18. An ultrasound apparatus according to claim 12 wherein the stand portion comprises a transducer port connected to the transducer connector, the transducer port configured to receive a transducer interface at an end of the transducer cord opposite the transducer.

19. A method of upgrading an ultrasound system comprising a display, an input device and a transducer coupled to a data processing apparatus, the method comprising:
providing a housing apparatus comprising a stand portion defining an enclosure for removably receiving the data processing apparatus, the housing apparatus comprising a cover moveable between an open position wherein the enclosure is accessible and a closed position wherein the enclosure in inacessible;
moving the cover to the open position;
uncoupling the display, the input device and the transducer from the data processing apparatus;
removing the data processing apparatus from the enclosure;
inserting a different data processing apparatus into the enclosure;
coupling the display, the input device and the transducer to the different data processing apparatus; and,
moving the cover to the closed position.
